(19) United States
(12) Patent Application Publication
(10) Pub. No.: US 2007/0292913 A1
(43) Pub. Date: Dec. 20, 2007

(54) Novel Bacterial Expression System

(75) Inventors: Leendert W. Hamoen, Oxford (GB); Oscar Paul Kuipers, Groningen (NL); Johanna Cordula Lindner, Gorssel (NL)

Correspondence Address: INTERVET INC.;PATENT DEPARTMENT, PO BOX 318, MILLSBORO, DE, 19966-0318, US

(73) Assignee: AKZO NOBEL N.V., ARNHEM (NL)

(21) Appl. No.: 10/523,729

(22) PCT Filed: Aug. 1, 2003
(86) PCT No.: PCT/EP03/08506

§ 371 Date: Feb. 4, 2005

(30) Foreign Application Priority Data
Aug. 7, 2002 (EP) 02078248.8

(51) Int. Cl. C07K 14/32; C07H 21/04; C12P 21/06; C12N 15/74; C12N 1/21

(52) U.S. Cl. 435/69.1; 435/252.3; 435/471; 536/23.7; 530/350

(57) ABSTRACT

The present invention relates to expression cassettes suitable for the expression of a heterologous gene. Furthermore, the invention relates to recombinant plasmids comprising such expression cassettes. Also, the invention relates to host cells comprising such cassettes or plasmids. Another embodiment relates to promoters for use in an expression cassette and in heterologous expression systems. Finally, an embodiment relates to methods for the expression of a heterologous gene.

The present invention relates to expression cassettes, to recombinant plasmids comprising such expression cassettes, to host cells comprising such cassettes or recombinant plasmids, to promoters for use in an expression cassette and use in heterologous expression systems and to methods for the expression of a heterologous gene.

Bacterial heterologous expression systems are universally used for the expression of a variety of different heterologous genes. A heterologous gene is considered to be a gene that does not originate from Bacillus subtilis or, if originating from Bacillus subtills, is not or not solely under the control of its native promoter. It may be a gene of bacterial origin, found in either the bacterial cell that is also used as the host for the expression system (e.g. in the case that over-expression of a native gene is needed) or from another species. It may equally well be a gene of eukaryotic origin. Bacterial heterologous expression systems may have a few known disadvantages over eukaryotic heterologous expression systems, but for the expression of many heterologous genes of both prokaryotic and eukaryotic origin, they provide very versatile and inexpensive systems. With regard to the host cells of bacterial expression systems, a difference is made between the Gram-positive and Gram-negative bacteria. Gram-negative bacteria have both an inner and an outer membrane. The outer membrane sometimes interferes negatively with transportation of secreted proteins over the cell membrane. Gram-positive bacteria do suffer to a much lesser extent from this problem, because they lack the outer membrane. A few randomly chosen examples of Gram-positive bacteria suitable as host cell for heterologous expression systems are e.g. bacteria belonging to the genera Lactococcus, Lactobacillus, Leuconostoc, Pediococcus, Streptococcus, Enterococcus, Staphylococcus, Bacillus, Sarcina, Ruminococcus or Listeria.

A problem connected with all heterologous expression systems is the fact that (over)-production of the heterologous expression product frequently interferes in a negative way with the viability or growth characteristics of the host cell in which it is expressed. Thus, when choosing a bacterial expression system, it has to be decided first, if the heterologous protein to be expressed is detrimental or even toxic for the host cell. If this is the case, the heterologous expression system would preferably have the option of switching the heterologous expression on or off. But even if the heterologous protein to be expressed is known to be only slightly or even not toxic at all for the host cell, continuous expression of the heterologous gene after the moment that growth reaches the stationary phase would finally kill the host cell. In such a case, an expression system would be preferred that continuously synthesizes the desired heterologous protein, but only to a level that is directly linked to the growth rate of the bacterium: if the growth rate slows down or halts, the heterologous expression level should preferably slow down or halt as well.

It is an objective of the present invention to provide a highly versatile expression system that offers a solution to the problems indicated above.

Surprisingly, it was found now that two genes coding for Single-stranded DNA Binding Proteins (SSB) found in i.a. the B. subtilis genome have promoter sequences that do have the characteristics that make them very suitable for a heterologous expression system with one or both of the desired characteristics.

One SSB-like gene, further referred to as ssb, maps at 358.6° of the B. subtilis genome and is flanked by the rpsF and rpsR genes coding for the ribosomal proteins S6 and S18, respectively (FIG. 1A). A structure similar to Rho-independent transcriptional terminators is situated downstream of the rpsR gene. Since no terminator-like structure was found between the genes rpsF, ssb and rpsR, it is likely that these genes belong to an operon. The second ssb-like gene, ywpH, maps at 319.4° and is flanked by another gene of unknown function (ywpG) and the gIcR gene coding for a regulator involved in carbon catabolite repression (FIG. 1B). Full details of the location and the sequence of the genes mentioned have been given in F. Kunst et al., Nature 390: 249-256 (1997).

Transcriptional fusions of the potential promoter-containing fragments with a promoter less lacZ gene were constructed and integrated into the B. subtilis chromosome at an ectopic site. An ectopic site is a site at which the gene is not found in the wild-type bacterium. Upstream regions of the ssb and ywpH genes, indicated as S 1, S2, R1 and Y1 were amplified by PCR using chromosomal DNA of B. subtilis 168 as template. The PCR fragments were cloned into a promoter-screening vector and introduced via transformation into B. subtilis 168, selecting for kanamycin resistant transformants. The transformants were screened for an amylase-deficient phenotype to confirm that the construct had integrated at the selected ectopic site, i.e. the amyE locus. (The amyE locus is merely an example of a suitable integration site, because the amyE gene is a non-essential gene, and can thus safely be used for the insertion of another gene.

The blue or white phenotypes of the resulting strains BIV7 (S1), 17 (S2), 8 (RI), and 12 (Y1) on rich (TY) and minimal medium (MM) agar plates revealed promoter activity only for the constructs in strain BIV 17, 8, 12 and 13, but not in BIV7. In BIV12, promoter activity was detected only in MM indicating a medium-dependent expression of the gene ywpH (FIG. 1). Whereas ywpH was found to be transcribed from a promoter directly upstream of the gene, no promoter activity could be detected from the S 1 fragment comprising the 295 bp immediately upstream of the ssb start codon. However, promoter activity was detected from the S2 fragment containing also the complete rpsF gene and 274 bp upstream of its start codon indicating that ssb is the second gene of an operon, which is constituted of the rpsF, ssb, and rpsR genes. Clearly promoter activity was found also from the smaller R1 fragment comprising a 274 bp upstream of the rpsF start codon only. These results indicate that ssb is cotranscribed and co-regulated with genes coding for ribosomal proteins, thereby coupling the regulation of protein synthesis and DNA metabolism. In order to study the expression of ssb and ywpH in more detail, strains containing the S1-, R1- and Y1-lacZ fusions (BIV7, 8 and 12), respectively, were grown in TY and MM and β-galactosidase activity was examined as described by Miller et al. (Miller, J. H. 1982. Experiments in molecular genetics. Cold Spring Harbor Laboratory Press, Cold Spring Harbor, N.Y.). Whereas no expression could be detected from the S1 fragment under the conditions employed, the ssb operon appeared to be strongly expressed from the rpsF-promoter in both rich and minimal media. The highest values (between 200 and 300 Miller units per OD) were reached during exponential growth (FIGS. 2A, B). When cells stopped growing the expression dropped. There is still expression in the stationary phase but on a much lower level.

In conclusion these results show directly growth-related characteristics of expression behavior of the ssb operon, and thus of the rpsF-promoter. Therefore, this promoter clearly is the promoter of choice for the expression of heterologous genes encoding non-toxic or slightly toxic proteins. The level of expression is directly linked to the growth rate of the bacterium. The protein encoded by the heterologous gene will be expressed only during logarithmic growth. If the growth rate of the bacterium slows down, e.g. as a result of decreased amounts of nutrients, the synthesis of the protein encoded by the heterologous gene also decreases, thus avoiding growth retardation or even killing of the host cell, i.e. the bacterium, by over-production of the heterologous gene.

One of the advantages of such an expression system is, that the host cells remain viable. Further expression can very easily be obtained by just adding more medium to the cells, regardless the fact that the cells may have temporarily reached at any moment in time the stationary phase.

In contrast, no expression of the ywpH gene could be detected in cells of B. subtils BIV12 grown in TY and only very low expression was observed in exponentially growing cells in MM. However, expression of ywpH increased significantly, when cells growing in MM entered the stationary phase and reached its highest level within two hours thereafter (FIG. 2C).

In conclusion: the ywpH promoter is only expressed during the stationary phase in MM. This promoter would clearly be the promoter of choice for the production of proteins that are toxic for the host cell, because during logarithmic growth of the cells, growth is not at all hampered by expression of toxic heterologous protein. Genes encoding such proteins can safely be cloned downstream of the ywpH promoter since the cells can be grown in MM or rich medium to a high density without any heterologous gene expression. Only as soon as the stationary phase is reached and cells are grown in MM, the heterologous toxic protein will be synthesized. Even more efficiently, cells can be grown in rich medium to high densities, i.e. the stationary phase, followed by further culturing in MM. This will then trigger the synthesis of the toxic protein. If needed, a small sample of cells can be taken out during logarithmic growth phase and kept frozen for use in further cultures.

The basic element of a heterologous expression system is the combination of the promoter and the heterologous gene to be expressed. This combination is in principle sufficient for obtaining the heterologous expression product, i.e. the protein encoded by the heterologous gene, as expressed under the influence of the rpsF-promoter or the ywpH-promoter.

This basic element will be further referred to as an expression cassette.

It should be clear however, that usually this expression cassette will subsequently be cloned into a plasmid, and/or be inserted in the genome of the host.

The recombinant plasmid carrying the expression cassette can be used for the transformation of the host bacterium. If a high-copy plasmid is chosen, this will generally lead to an enhanced expression, due to the fact that several copies of the expression cassette will be expressed at the same time in the host. This allows for a further fine-tuning of the amount of expression product made. Such high-copy plasmids are known in the art. Instead of maintaining the expression cassette on a plasmid in the host, the expression cassette can be integrated, in one or more copies, in the genome of the host cell, e.g. through the process of homologous recombination. A combination of the two systems is also feasible.

Thus a first embodiment of the present invention relates to an expression cassette comprising a promoter which is obtainable from the Bacillus subtilis genome wherein the promoter is located in the region between the stop codon of the yyaF-gene and the start codon of the rpsF-gene.

Only one promoter is found in this region, and therefore the fact that the promoter according to the invention is located in the region between the stop codon of the yyaF-gene and the start codon of the rpsF-gene is an unequivocal identification of the promoter.

As mentioned above, full details of the location and the sequence of the genes mentioned here have been given in F. Kunst et al., Nature 390: 249-256 (1997).

Genomes of microorganisms whose complete genome sequence is known were screened for SSB homologues and their gene organization using the NCBI database (http://www.ncbi.nhn.nih.gov). At this moment 69 complete sequences of bacterial genomes are available including 59 different species. Of these, 23 species show the ssb gene flanked by the rpsF and rpsR genes as was observed for B. subtilis. These species mainly belong to the taxonomic group of the Firmicutes, like B. subtilis itself, to the Spirochaetales, Thermotogales and Aquificales.

This search revealed that the gene organization rpsF ssb rpsR is commonly found in these taxonomic groups.

The sequence of this region in Bacillus subtilis is given in FIG. 3. The desired promoter region, which is described in more detail below, can now easily be found on the genome of Bacillus subtilis, and subsequently be synthesized and cloned with the help of standard PCR techniques, well-known in the art. Such PCR- and cloning techniques are e.g. described in standard laboratory manuals such as in Dieffenbach & Dreksler; PCR primers, a laboratory manual. ISBN 0-87969-447-5 (1995) and in Maniatis/Sambrook (Sambrook, J. Molecular cloning: a laboratory manual, 1989. ISBN 0-87969-309-6).

The promoter of the expression cassette according to the present invention is obtainable from Bacillus subtilis as mentioned above, but was shown, as also stated above, to be present in the taxonomic group of the Firmicutes, like B. subtilis itself, the Spirochaetales, Thermotogales and Aquificales. It can therefore easily be located and cloned from these taxons by screening the genome of any of the bacteria belonging to these taxons, using the sequence given in FIG. 3 as a probe. Such screening is also a standard technique, known in the art for years now, and also described in Dieffenbach & Dreksler, PCR primers, a laboratory manual. ISBN 0-87969-447-5 (1995) and in Maniatis/Sambrook (Sambrook, J. Molecular cloning: a laboratory manual, 1989. ISBN 0-87969-309-6).

Therefore, promoters as described in the invention obtained from any of the taxonomic groups mentioned above are considered to be within the scope of the present invention.

It is clear that any of the taxonomic groups mentioned above are also suitable for use as a host cell for expression cassettes according to the invention, because the promoter is present in, and thus recognized in these taxonomic groups.

Promoter sequences can be recognized by a few motives, such as their specific so-called −35 site, −10 site and the spacer between these sites, as well as the fact that at a certain distance from these sites of (very roughly) 50-65 nucleotides downstream of the −10 site a ribosome binding site motive is found. The distance between −10 site and RBS is however far from critical.

In a preferred form of this embodiment, the rpsF promoter of the heterologous expression system according to the invention has the following characteristics:
a) a nucleotide sequence TATAAT, preferably GTATAAT, TATAATA or GTATAATA, more preferably GGTATAAT, TATAATAT or GGTATAATAT at the −10 site,
b) a nucleotide sequence TTGTAA, preferably GTTGTAA, TTGTAA or GTTGTAAA, more preferably AGTTGTAA, TTGTAAAG or AGTTGTAAAG at the −35 site,
c) a spacer of 17+/−2 nucleotides, preferably 17+/−1 nucleotides, more preferably 17 nucleotides.

A spacer is understood to be the nucleic acid sequence separating the −35 sequence TTGTAA from the −10 sequence TATAAT. This spacer varies in length between 15 and 19 nucleotides.

In a more preferred form of this embodiment, the promoter comprises a nucleotide sequence as depicted in SEQ ID NO 1.

In principle, the Ribosome Binding Site (RBS) to be used is not critical, especially because it is not an actual part of the promoter as such. Nevertheless, the RBS as found between the rpsF-promoter and the start codon of the rpsF gene would be a preferred RBS.

Therefore, in an even more preferred form, the promoter comprises a nucleotide sequence as depicted in SEQ ID NO 2. This sequence differs from the sequence of SEQ ID NO 1 in that it additionally comprises the sequence downstream of the −10 region to include the Ribosome Binding Site (RBS) preceding the rpsF gene.

In order to obtain the most native form of the rpsF promoter, it is preferred to also use the region between the RBS and the start-codon (preferably including the start codon) of the rpsF gene.

Thus, in a most preferred form, the promoter according to the invention comprises a nucleotide sequence as depicted in SEQ ID NO 3. This sequence differs from the sequence of SEQ ID NO 2 in that it additionally comprises the sequence downstream of the RBS to include the ATG-codon of the rpsF gene.

As can be seen from FIG. 3, the DNA sequence located upstream of the −35 region comprises several AT-rich regions which will render the DNA helix more flexible. This recurrent motive is a typical DNA bending motive.

Such DNA-bending motives make it possible for distant upstream sequences involved in transcription, to become spatially located close to the promoter region.

As shown in FIG. 5, several constructs have been made, in which the rpsF-promoter according to the invention is fused to a promoter-less β-galactosidase gene, and is preceded by stretches of nucleotides, of different length, as present upstream of the promoter sequence in the native situation. FIG. 6 clearly shows that those constructs having longer stretches of native nucleotide sequences upstream the −35 region still in place give a higher expression level compared to those constructs having a shorter upstream −35 sequence.

This clearly shows, that the presence of these AT-rich regions enhances the efficiency of the rpsF-promoter according to the invention.

Therefore, in a more preferred form of this embodiment, the promoter described in the invention is preceded by a stretch of nucleotides having at least 1, preferably 2, more preferably even three AT-rich regions, such as e.g. ATTT, ATTTA or TTATT upstream of the −35 region.

Preferably, the stretch of nucleotides having AT-rich regions upstream of the −35 region comprises the nucleotide sequence as depicted in SEQ ID NO 4, more preferably SEQ ID NO 5, even more preferably SEQ ID NO 6.

The sequence of the ywpH promoter is given in FIG. 4. Actually, the region of this promoter that is of importance is referred to as a comK-box. ComK-boxes play an important role in the competence-signal-transduction network. ComK-boxes have as a common characteristic that they share the consensus motive AAAANNNNNTTTT. The following must be stressed here: a consensus motive is a motive statistically the most prevalent motive found, when several of these motives are compared. This does not mean that minor modifications are not tolerated. Occasionally, A/T-, A/G- or A/C-transitions are found in the AAAA stretch of the motive and occasionally, T/A-, T/G- or T/C-transitions are found in the TTTT-stretch. The TATT tetranucleotide found instead of the TTTT sequence in the most upstream located comK-box of the ywpH gene is a good example of such a transition. Therefore, a “comK-box” is understood to have a sequence that does not differ at the “A”-side and/or the “T”-side of the box in more than one nucleotide compared to the consensus sequence. As a result, a comK-box as used in the present invention, i.e. falling under the definition of a consensus comK-box, could e.g. have the sequence AAAANNNNNTTTT, ATAANNNNNTTTT, AATANNNNNTATT or AAAANNNNNTATT. These examples are of course not limiting.

Another characteristic of comK-boxes is that they are always found twice. The presence of two comK-boxes is a prerequisite for their functionality.

The two comK-boxes in FIG. 4 are presented in bold letters.

The length of the spacer region separating the two comK-boxes is not critical. Values between 10 and 150 base pairs are however quite commonly found.

The two comK-boxes are located upstream of the gene to be expressed at a distance that is not critical. Roughly however, the distance between the comK-boxes and the gene to be expressed should not exceed 500 nucleotides. Typically, the distance would be between 20 and 200 nucleotides.

More information about the comK-boxes can be found e.g. at http://elmo.ims.u-tokyo.ac.ip/dbtbs/dfac/ComK.html.

Therefore, another embodiment of the invention relates to an expression cassette that comprises a promoter having two comK-boxes with a consensus sequence AAAANNNNNTTTT that are present twice within a mutual distance of between 10 and 150 nucleotides. Thus the sequence of this kind of promoter can be represented shortly as follows: AAAANNNNNTTTTXₙAAAANNNNNTTT, wherein Xₙ represents a number of nucleotides between 10 and 150, and N and X can be A, T, G or C.

In a preferred form of this embodiment, the expression cassette according to the invention comprises the promoter of the ywpH gene.

This promoter comprises the nucleotide sequence as depicted in SEQ ID NO: 7.

In a more preferred form of this embodiment, the promoter is followed by the native downstream nucleotide sequence up to, and including, the ywpH RBS. This promoter has the sequence as depicted in SEQ ID NO: 8.

In an even more preferred form of this embodiment, the promoter is followed by the native downstream nucleotide sequence up to, and including, the ywpH RBS and further extending to the start codon of the ywpH gene. This promoter has the sequence as depicted in SEQ ID NO: 9.

Another embodiment of the invention relates to recombinant plasmids that comprise an expression cassette according to the invention. As mentioned above, such plasmids serve as a way of transforming the host cell. Such plasmids may be high-copy or low-copy plasmids.

Techniques for the transformation of bacterial cells, more specifically Bacillus-like cells are well-known in the art.

If desired, 5′-terminal and 3′-terminal parts of a bacterial gene encoding an e.g. non-essential gene can be added at the upstream and downstream end of the expression cassette according to the invention. This allows for homologous recombination with the host cell's genome once the expression cassette optionally carried by the recombinant plasmid is brought into the host cell. The expression cassette will thus become stably integrated into the host's genome. Homologous recombination into a selectable non-essential gene highly facilitates the selection for integrants. As a selectable non-essential gene in B. subtilis, the amyE gene would be a very suitable gene.

Still another embodiment of the invention relates to bacterial host cells comprising an expression cassette or a recombinant plasmid according to the invention. As mentioned above, this embodiment relates to both bacterial host cells comprising an expression cassette located on a recombinant plasmid and bacterial host cells comprising an expression cassette integrated in the bacterial genome.

Again still another embodiment of the invention relates to promoters as described above for use in an expression cassette.

And still another embodiment of the invention relates to promoters as described above for use in a heterologous expression system.

Again another embodiment of the invention relates to methods for the heterologous expression of a gene wherein those methods comprise the construction of an expression cassette according to the present invention. Examples of the construction of such an expression cassette are given in the Examples below.

Still other embodiments relate to the use of a promoter as described in the present invention in an expression cassette and to the use of such a promoter in a heterologous expression system.

Finally, an embodiment of the present invention relates to methods for the heterologous expression of a gene, that comprise the transformation of a bacterium with an expression cassette according to the invention.

### EXAMPLE 1

### Transcription Analysis of the ssb and ywpH Genes

For all PCR-reactions B. subtilis 168 DNA was used as template. This strain can be obtained from the Bacillus Genetic Stock Center; Strains & Data, The Ohio State University, Department of Biochemistry, 484 West Twelfth Avenue, Columbus, Ohio 43210, USA.

Expression of these two genes and localization of their respective promoters. Transcriptional fusions of the potential promoter-containing fragments with a promoter less lacZ gene were constructed and integrated into the B. subtilis chromosome at an ectopic site: the alpha-Amylase E gene, amyE.

Upstream regions of the ssb and ywpH genes, fragment S1 (from 295 bp upstream to 11 bp downstream of the start codon of ssb), S2 (from 602 bp upstream to 11 bp downstream of the start codon of ssb), R1 (from 274 bp upstream to 50 bp downstream of the start codon of rpsF), Y1 (from 282 bp upstream to 10 bp downstream of the start codon of ywpH) were amplified by PCR using Pwo DNA polymerase (Roche) and chromosomal DNA of B. subtilis 168 as template. The PCR fragments were cloned into the SmaI-digested promoter-screening vector pBTK2 (pBTK2 is a plasmid that carries 3′- and 5′-terminal parts of the Bacillus subtilis amyE gene, each flanking one side of a promoter less lacZ gene. See for details: Meijer, W. J. J. 1995. Replication and maintenance of plasmids in Bacillus subtilis. Thesis, available through the University of Groningen, The Netherlands). Resulting plasmids carrying the insert in the correct orientation were linearized and used to transform B. subtilis 168, selecting for kanamycin resistant transformants. The transformants were screened for an amylase-deficient phenotype to confirm that the construct had integrated at the amyE locus. The cloning and transformation were performed according to common techniques (Sambrook, J., E. F. Fritsch, and T. Maniatis. 1989. Molecular cloning: A Laboratory Manual. Cold Spring Harbor Laboratory Press, Cold Spring Harbor, N.Y., the supplier's manuals, and Bron, S, and G. Venema. 1972. Mutat. Res. 15:1-10).

The blue or white phenotypes of the resulting strains BIV7 (S1), 17 (S2), 8 (RI), and 12 (Y1) on rich (TY) and minimal medium (MM) agar plates (Meima, R., C. Eschevins, S. Fillinger, A. Bolhuis, L. W. Hamoen, R. Dorenbos, W. J. Quax, J. M. van Dijl, R. Prowedi, 1. Chen, D. Dubnau, and S. Bron. 2002. J. Biol. Chem. 277: 6994-7001), respectively, containing 0.004% X-Gal revealed promoter activity only for the constructs in strain BIV 17, 8, 12 and 13, but not in BIV7. In BIV12, promoter activity was detected only in MM indicating a medium-dependent expression of the gene ywpH (FIG. 1). Whereas ywpH was found to be transcribed from a promoter directly upstream of the gene, no promoter activity could be detected from the S 1 fragment comprising the 295 bp immediately upstream of the ssb start codon. However, promoter activity was detected from the S2 fragment containing also the complete rpsF gene and 274 bp upstream of its start codon indicating that ssb is the second gene of an operon, which is constituted of the ipsF, ssb, and ipsR genes.

Clearly promoter activity was found also from the smaller R1 fragment comprising the 274 bp upstream of the rpsF start codon only. These results suggest that ssb is cotranscribed and co-regulated with genes coding for ribosomal proteins, thereby coupling the regulation of protein synthesis and DNA metabolism.

In order to study the expression of ssb and ywpH in more detail, strains containing the S1-, R1- and Y1-lacZ fusions (BIV7, 8 and 12), respectively, were grown in TY and MM and β-galactosidase activity was examined as described by Miller et al. (Miller, J. H. 1982. Experiments in molecular genetics. Cold Spring Harbor Laboratory Press, Cold Spring Harbor, N.Y.). Whereas no expression could be detected from the S1 fragment under the conditions employed, the ssb operon appeared to be strongly expressed from the rpsf-promoter in both rich and minimal media. The highest values (between 200 and 300 Miller units per OD) were reached during exponential growth (FIGS. 2A, B). When cells stopped growing the expression dropped. There is still expression in the stationary phase but on a much lower level.

In conclusion these results show directly growth-related characteristics of expression behavior of the ssb operon, and thus of the rpsF-promoter.

In contrast, no expression of the ywpH gene could be detected in cells of B. subtilis BIV12 grown in TY and only very low expression was observed in exponentially growing cells in MM. However, expression of ywpH increased significantly, when cells growing in MM entered the stationary phase and reached its highest level within two hours thereafter (FIG. 2C).

In conclusion: the ywpH promoter is only expressed during the stationary phase in MM.

Below, the primers for, and the sequences of the S1, S2, R1 and Y1 constructs are given. Underlined regions reflect restriction sites.

S1: made with primers

ssb-1 Forward primer to amplify the ssb promoter region GCGAAGCTTCCAAACATTGACGAAGAGTCT

ssb-2 Reverse primer to amplify the ssb promoter region GCTGGATCCTCGGTTAAGCATAAGAAGACC

Sequence:

GCTTCCAAACATTGACGAAGAGTCTAAAAAAGCAGTTATCGAGCGTTTCA
ACAACGTTCTGACTTCTAACGGTGCGGAGATCACTGGAACAAAGGATTGG
GGTAAACGTCGTCTTGCTTACGAAATCAACGATTTCCGCGACGGTTTCTA
CCAAATCGTAAACGTTCAATCTGACGCTGCGGCAGTTCAAGAATTTGACC
GTCTAGCTAAGATCAGTGACGATATCATTCGCCACATTGTTGTTAAAGAA
GAAGAATAAGCAATTTTGAAATATATAATGGTAAAAGGTGGTCTTTCTTA
TGCTTAACCGAGGAT

S2: made with primers
rps-1 Forward primer to amplify the rpsF promoter region GCGAAGCTTGTGACTTTGAGCGGGGCTT
Ssb-2 Reverse primer to amplify the ssb promoter region GCTGGATCCTCGGTTAAGCATAAGAAAGACC

Sequence:

GCTTGTGACTTTGAGCGGGGCTTCATTCGTGCTGAGACAGTTGCTTATGA
GGATCTTCTTGCGGGCGGCGGTATGGCAGGAGCTAAAGAGGCAGGAAAAG
TCCGCCTTGAAGGGAAAGAATATGTGGTCCAAGACGGAGATGTTATTCAT
TTCCGATTTAATGTATAGGATGCAGTTGTAAAGGGACAAGAGCTTTGGTA
TAATATAAAATTGTGAGTAATAGAATTATTGCTCCTTGCCCATTATGGGC
CGCTTAGTCCAAAAGGAGGTGCAAACAGATGAGAAAGTACGAAGTTATGT
ACATTATCCGCCCAAACATTGACGAAGAGTCTAAAAAAGCAGTTATCGAG
CGTTTCAACAACGTTCTGACTTCTAACGGTGCGGAGATCACTGGAACAAA
GGATTGGGGTAAACGTCGTCTTGCTTACGAAATCAACGATTTCCGCGACG
GTTTCTACCAAATCGTAAACGTTCAATCTGACGCTGCGGCAGTTCAAGAA
TTTGACCGTCTAGCTAAGATCAGTGACGATATCATTCGCCACATTGTTGT
TAAAGAAGAAGAATAAGCAATTTTGAAATATATAATGGTAAAAGGTGGTC
TTTCTTATGCTTAACCGAGGAT

R1: made with primers:
Rps-1 Forward primer to amplify the rpsF promoter region GCGAAGCTTGTGACTTTGAGCGGGGCTT
Rps-2 Reverse primer to amplify the rpsF promoter region GCTGGATCCATCTTCGTCAATGTTTGGGCG

Sequence:

GCTTGTGACTTTGAGCGGGGCTTCATTCGTGCTGAGACAGTTGCTTATGA
GGATCTTCTTGCGGGCGGCGGTATGGCAGGAGCTAAAGAGGCAGGAAAAG
TCCGCCTTGAAGGGAAAGAATATGTGGTCCAAGACGGAGATGTTATTCAT
TTCCGATTTAATGTATAGGATGCAGTTGTAAAGGGACAAGAGCTTTGGTA
TAATATAAAATTGTGAGTAATAGAATTATTGCTCCTTGCCCATTATGGGC
CGCTTAGTCCAAAAGGAGGTGCAAACAGATGAGAAAGTACGAAGTTATGT
ACATTATCCGCCCAAACATTGACGAAGATGG

Y1: made with primers
ywpH-1 Forward primer to amplify the ywpH promoter region CCCAAGCTTTCAAGCTGTCAATGCCG
ywpH-2 Reverse primer to amplify the ywpH promoter region CGCGGATCCGATTGAACATGCGATTCC

Sequence:

GCTTTCAAGCTGTCAATGCCGGAAAAAAAATTGAGCTTTCAGTGGTTTGC
GTGGGATGGCTCTTCCTATGTGCGCATGAATACGCAAAACTGGCTGACAA
AGCAAATCTTTTTCCGTTTTTTAAAAAGTACATATTTCTTCAAAGGAAAA
AAGCAAAAGATGTTTTTAGCTGAAGGAAAAATGAAAACGAAAGATAAAAA
CAGAGGCTGAAAGCCATTTTTAAGCGTTTTTCTTTTCTTGTTGCATCATT
TACAATACATACAACCGCAAGGAGAGGAGGAATCGCATGTTCAATCGGAT

### EXAMPLE 2

### Cloning Strategy Bacillus subtilis Integration/Multi Copy Plasmid with Expression Cassette

In this construct, the rpsF promoter as described in the invention is used, but the RBS is the spoVG RBS as also found on the Bacillus subtilis genome. The gene cloned and expressed in this expression cassette is the Clostridium perfringens beta-toxin (Hunter et al.; Infect. & Immun. 61: 398-965 (1993))

1 PCR Reactions:

1.1 amyE

Primers for amplifying the amyE gene introducing a 5′ XbaI site and a 3′ SacI site were as follows:

amyE-1:
Forward primer: GCTCTAGACGAATTATATGGATGTGAC
amyE-2:
Reverse primer: GCCTAGAGCTCGGATCTCCTTTTCCGATTG

Template: chromosomal DNA 168
Product size: 823 bp
1.2 Kanamycin Resistance Gene

Primers for amplifying the kanamycin resistance gene (same as in pUB110) introducing 5′ PstI and NruI sites and a 3′ kpnI site

Kn-1:
Forward primer: CAATCTGCAGTCGCGATGAGAATAGTGAATGGAC
Kn-2:
Reverse primer: CGGGTACCTCAAAATGGTATGCGTTTTG

Template: pUB110 (Gryczan, T. J., Contente, S., Dubnau, D.; J. Bacteriol. 134: 318-329 (1978), McKenzie, T., Hoshino, T., Tanaka, T., Sueoka, N.; Plasmid 15: 93-103 (1986)).
Product size: 794 bp
1.3 spoVG RBS

Artificially made:

GAAGATCTCTCGAGGGTACCTTGATACACTAATGCTTTTATATAGGGAAA
AGGTGGTGAACTCATATGAATCGAGCTTCTAGAGAGCTCCCATGGAACTA
TGTGATTACGAAAATATATCCCTTTTCCACCACTTGAGTATACTTAGCTC
GAAGATCTCTCGAGGGTACCTTGATACAC CTAATGCTTTTATATAGGGA
AAAGGTGGTGCATGGAACTATGTGATTACGAAAATATATC CCTTTTCCA
CCACTTGAGTATACTTAGCTC

Primers:

spoVG1 5′-GAAGATCTCTCGAGGGTACCTTGATACAC-3′
spoVG2 5′-CTATATAAAAGCATTAGTGTATCAAGGTAC-3′
spoVG3 5′-CTAATGCTTTTATATAGGGAAAAGGTGGTG-3′
spoVG4 5′-CTCGATTCATATGAGTTCACCACCTTTTCC-3′

spoVG RBS can be found on the Bacillus subtilis genome.

Because of a BglII site in this kanamycin gene, the spoVG RBS could not be cloned with BglII, so instead of BglII a PstI site must be introduced:

spoVG5:
Forward primer: GACTGCAGCTCGAGGGTACCTTGATACAC
spoVG4:
Reverse primer: CTCGATTCATATGAGTTCACCACCTTTTCC

Template: spoVG RBS
Product size: 75 bp
1.4 gnt Terminator

Primers for amplifying 3 stop codons and the gnt-terminator introducing 5′ BamHI and HpaI sites and 3′ XbaI and StuI sites.

Gnnterm-1:
Forward primer: GCGGATCCAGGCCTAACTAATTAACCTGTATTAA
AAACACG
Gntterm-2:
Reverse primer: GCTCTAGAGTTAACCTTCTGTTGTTTGGGATAG

Template: chromosomal DNA 168
Product size: 107 bp.
1.5 Clostridium Perfringens Beta-Toxin (Btox)

Primers for amplifying Btox, introducing a 5′ PstI, NdeI site and a 3′ BamHI site.

B-toxoid3:
5′-AACTGCAGAGATCTCATATGAAGAAAAAATTTATTTCATT-3′
Btoxoid2:
CGCGGATCCTTAAATAGCTGTTACTTTGTGAG

Product size: About 1000 bp

1.6 The rpsF promoter without rpsF RBS

rpsF-3:
5′-GGAATTCCTGCAGGTGACTTTGAGCGGGGCTTC-3′
rpsF-4:
5′-CGTACTTTCTCATATGTTTGCACC-3′

Template: chromosomal DNA 168
Product size: 295 bp.
2 Cloning:
2.1 Cloning Kanamycin in pBlueSK-

The kanamycin PCR product was digested with PstI and KpnI and ligated into the likewise digested pBlueSK-. This resulted in plV26.

2.2 Cloning amyE in plV26

The amyE PCR product was subsequently digested with XbaI and SacI and ligated into the likewise digested plV26. This resulted in plV27.

2.3 Cloning Btox In plV27

The Btox PCR product was digested with PstI and BamHI and ligated into the likewise digested plV27. This resulted in plV28.

2.4 Cloning the gnt Terminator in plV28

The gnt terminator PCR fragment was digested with XbaI and BamHI and ligated into likewise digested plV28, which resulted in plV29.

2.5 Cloning the spoVG RBS in plV29

The spoVG PCR product was digested with PstI and NdeI and ligated into the likewise digested plV29. This resulted in plV33.

2.6 Cloning the spoVG-RBS+Btox+Terminator−Cassettes in pTRKH2

Plasmid plV33 was digested with XbaI and PstI and ligated into likewise digested pTRKH2. This resulted in plV69. pTRKH2 is described by O'Sullivan, D. J. and Klaenhammer, T. R. in Gene 137: 227-231 (1993).

2.7 Cloning the rpsF Promoter (with spoVG RBS) in plV69

Plasmid plV69 was digested with StuI and ligated to the blunt ended rpsF PCR fragment. This resulted in plV82.

This plasmid was used for transformation of Bacillus subtilis.

Bacillus subtilis was subsequently transformed with the construct of 2.7, and grown in rich medium. As is clear from FIG. 7, large amounts of Clostridium perfringens beta-toxin were expressed and excreted into the supernatant. The increase in formation of insoluble material in the cell is probably due to too high a level of expression. The expression level can easily be decreased by using a low copy vector, or by integration of the expression cassette in the genome at a single integration site or at a few integration sites.

So far, several other toxins, i.a. the Pasteurella multocida toxin (see i.a. EP409895), the E. coli heat-labile toxin and the Staphylococcus aureus alfa-toxin have equally successfully been expressed.

This shows that the expression cassettes according to the invention are very versatile expression vectors.

### EXAMPLE 3

### Cloning Strategy Double Integration Construct

1. PCR

1.1 amyEback:

PCR for amplifying the 5′ amyE region, introducing a HindIII and a XhoI site.

amyEback-1:
CCCAAGCTTTCGACATGGATGAGCGATG
amyEback-2:
GCAGCTCGAGGCTCCGGCGCAAATGCAG

Product size: 1256 bp
1.2 amyEfront:

PCR for amplifying the 3′ amyE region, introducing a SacI and a XbaI site.

amyEfront-1:
CGCGAGCTCAACAAAATTCTCCAGTCTTC
amyEfront-2:
CGGTCTAGAAGTTTTTAATTTGTGTGTTTCC

Product size: 634 bp
1.3 Chloramphenicol:

PCR for amplifying the chloramphenicol resistance gene, introducing a EcoRI and a HindIII site.

cmR-1:
CGGGAATTCTCATGTTTGACAGC
cmR-2:
CGCGAAGCTTCCCAGTAGTAGGTTGAGGCC

Product size: 1310 bp
1.4 Terminator:

PCR for amplifying stop codons in all frames and the gnt terminator sequence, introducing BamHI, SnaBI, HpaI and XbaI sites.

Gntterm-3:
Forward primer: GCGGATCCTACGTAAACTAATTAACCTGTATTAA
AAACACG
Gntterm-2:
Reverse primer: GCTCTAGAGTTAACCTTCTGTTGTTTGGGATAG

Template: chromosomal DNA 168
Product size: 107 bp.
1.5 rpsF Promoter:

PCR for amplifying the rpsF promoter, introducing a PstI and a NdeI site.

rpsF-3:
5′-GGAATTCCTGCAGGTGACTTTGAGCGGGGCTTC-3′
rpsF-5
5′-CGTACTTTCTCATATGTTTGCACC-3′

Template: 168 chromosomal DNA
Product size: 295 bp
1.6 Btox:

PCR for amplifying Btox, introducing a BamHI and NdeI site.

B-toxoid3:
AACTGCAGAGATCTCATATGAAGAAAAAATTTATTTCATT
Btoxoid2:
CGCGGATCCTTAAATAGCTGTTACTTTGTGAG

Product size: 1037 bp
2 Cloning
2.1 amyEback in pBlueKS-

The amyEback PCR product was digested with HindIII and XhoI and ligated into the likewise digested pBlueKS— to produce construct 1.

2.2 amyEfront in Construct 1.

The amyEfront PCR product was digested with SacI and XbaI and ligated into likewise digested 1 to produce construct 2.

2.3 Chloramphenicol in Construct 2.

The cm PCR product was digested with EcoRI and HindIII and ligated in likewise digested 2 to produce construct 3.

2.4 gnt Terminator in Construct 3.

The terminator PCR product was digested with BamHI and XbaI and ligated in likewise digested 3 to produce construct 4.

2.5 rpsF Promoter In Construct 4.

The rpsF PCR product was digested with PstI and ligated into PstI-SmaI digested 4 to produce construct 5.

2.6 Btox in Construct 5.

The Btox PCR product was digested with BamHI and NdeI and ligated into likewise digested 5 to produce construct 6.

### LEGEND TO THE FIGURES

FIG. 1: Gene organization of the ssb operon (A) and the ywpH gene (B) in B. subtilis 168 with schematic representation of the constructed lacZ fusions. Black lines in bold type represent the PCR-amplified DNA fragments fused to the promoter less lacZ gene.

The presence or absence of 1-galactosidase activity in the various constructs after growth in rich (TY) or minimal medium (MM) is indicated on the right site by a ‘+’ and ‘−’, respectively.

FIG. 2: Expression of the ssb operon and the ywpH gene in B. subtilis.

Bacterial growth (open symbols) in TY (triangles) or minimal medium (circles) and expression of the transcriptional lacZ fusions (closed symbols) under the control of the rpsF promoter, in the strain BIV8 (A, B) or the ywpH promoter in the strain BIV12 (C) reflected as β-galactosidase activity per OD.

FIG. 3: upstream region of the rpsF gene, indicating the location of the −10 and −35 regions and AT-rich stretches further upstream, as well as the RBS and the start codon of the rpsF gene.

FIG. 4: upstream region of the ywpH gene, indicating the location of the two comK boxes (bold), the RBS (italic) and the 30 first nucleotides (including ATG start codon) of the ywpH gene (underlined).

FIG. 5: schematic representation of constructs of various length comprising the rpsF promoter fused to the f-galactosidase gene. The location of the −10 and −35 region, the RBS and the rpsF start codon as well as the length of the various upstream fragments are indicated.

FIG. 6: schematic representation of the β-galactosidase activity of the various constructs of FIG. 5.

FIG. 7: Expression of Btox under the control of rpsF promoter on a high copy vector. Sup=supernatant, Sol=soluble fraction, Insol=insoluble fraction. BtoxCp=wild type Clostridium perfringens beta-toxin. T=growth time in hours.

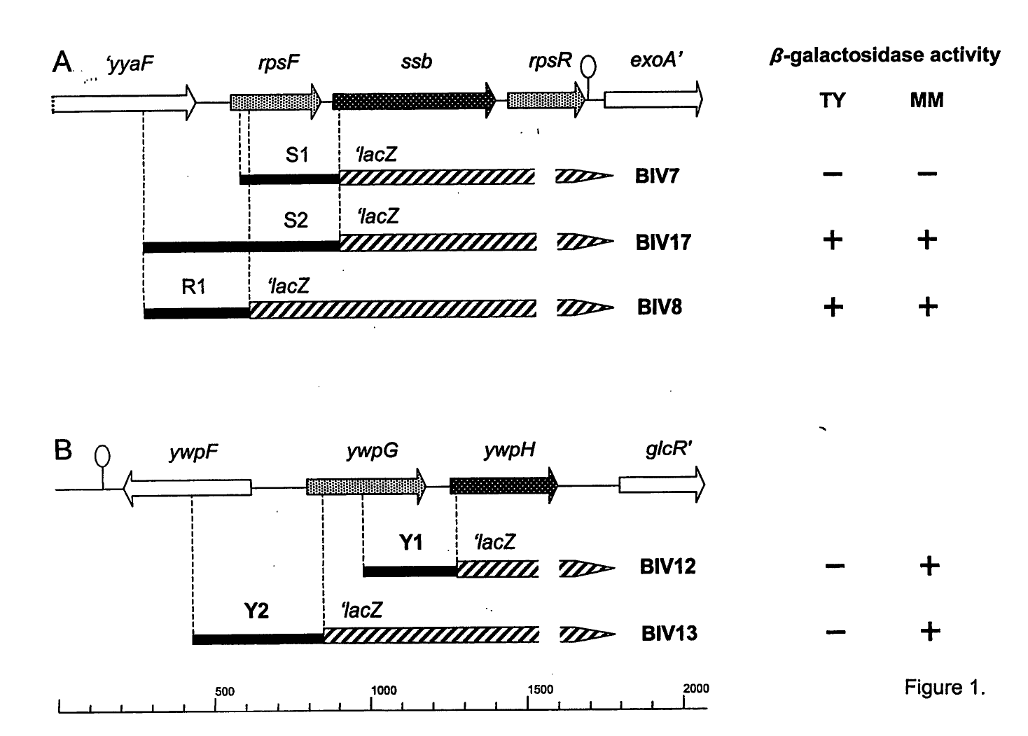

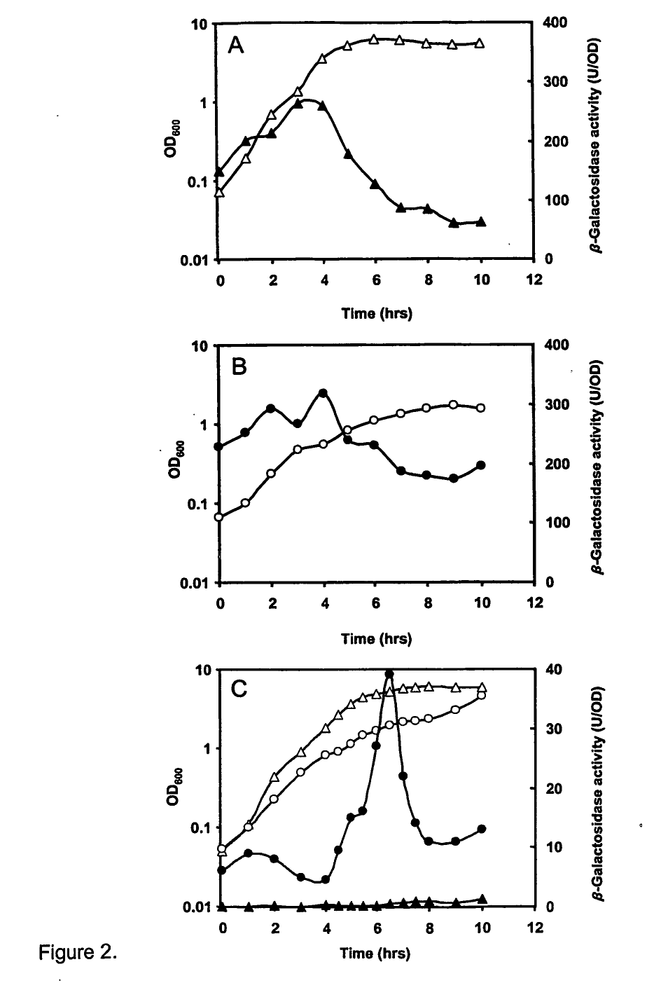

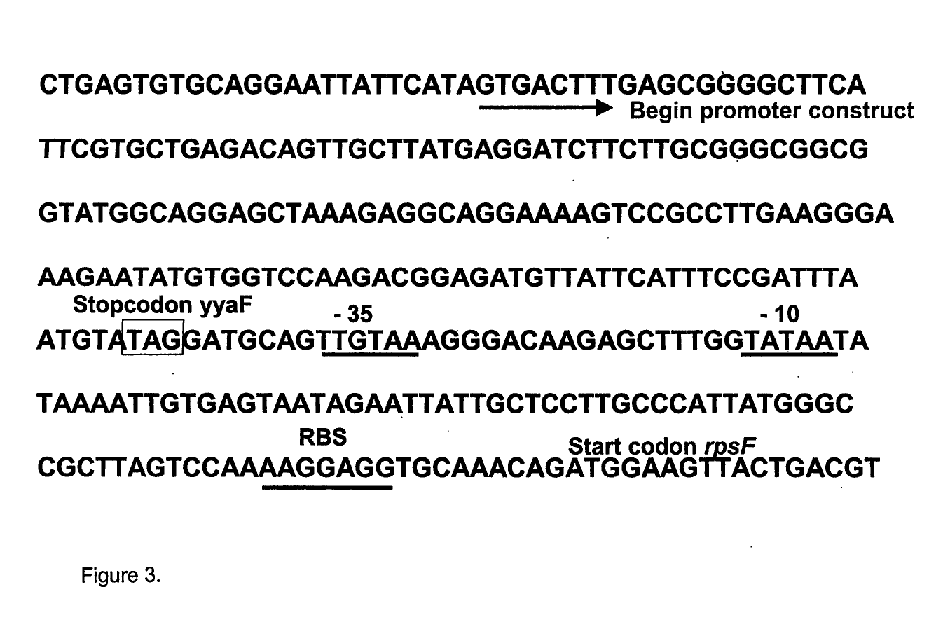

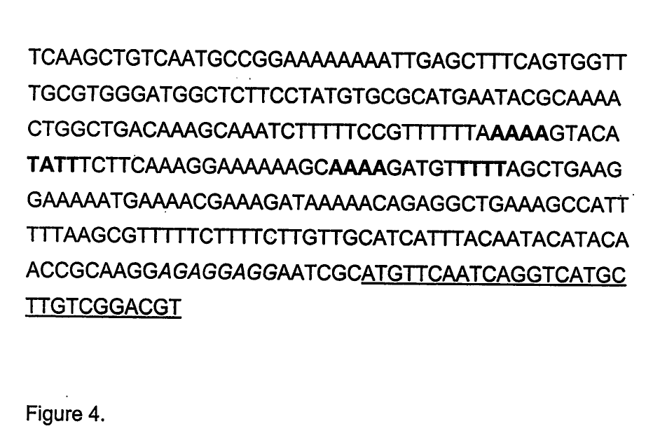

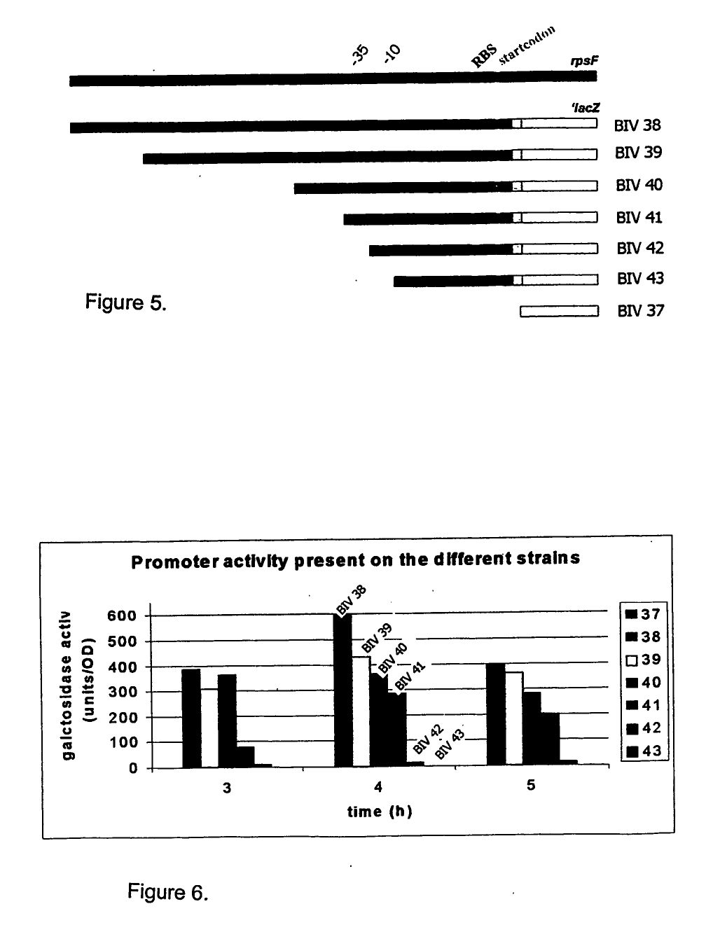

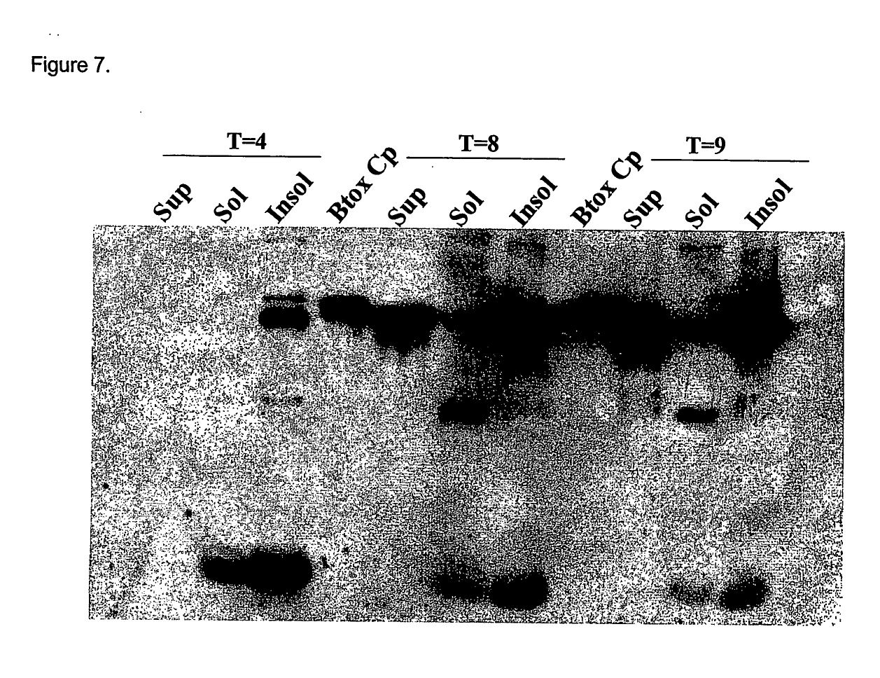

1. An expression cassette, characterized in that said expression cassette comprises a promoter obtainable from the region between the stop codon of the yyaF gene and the start codon of the rpsf gene as found on the Bacillus subtilis genome.

2. An expression cassette, characterized in that said expression cassette comprises a promoter having the following characteristics
a) a nucleotide sequence TATAAT, preferably GTATAAT or TATAATA at the −10 site,
b) a nucleotide sequence TTGTAA, preferably GTTGTAA or TTGTAAA at the −35 site,
c) a spacer of 17+/−2 nucleotides, preferably 17+/−1 nucleotides, more preferably 17 nucleotides.

3. The expression cassette according to claim 1, characterized in that the promoter comprises the nucleotide sequence as depicted in SEQ ID NO 1.

4. The expression cassette according to claim 1, characterized in that the promoter comprises the nucleotide sequence as depicted in SEQ ID NO 2.

5. The expression cassette according to claim 1, characterized in that the promoter comprises the nucleotide sequence as depicted in SEQ ID NO 3.

6. The Expression cassette according to claim 1, characterized in that the promoter is preceded by a stretch of nucleotides having at least 1, preferably 2, more preferably even three AT-rich regions upstream of the −35 region.

7. The expression cassette according to claim 6, characterized in that said stretch of nucleotides having AT-rich regions upstream of the −35 region comprises the nucleotide sequence as depicted in SEQ ID NO 4, preferably in SEQ ID NO 5, more preferably in SEQ ID NO 6.

8. An expression cassette, characterized in that said expression cassette comprises a promoter having a consensus sequence AAAANNNNNTTTTXₙAAAANNNNNTTTT (SEQ ID NO: 16), wherein 10<n<150.

9. The expression cassette according to claim 8, characterized in that the promoter comprises the nucleotide sequence as depicted in SEQ ID NO: 7, preferably in SEQ ID NO: 8, more preferably in SEQ ID NO: 9.

10. Recombinant plasmid characterized in that said plasmid comprises an expression cassette according to claim 1.

11. Bacterial host cell comprising an expression cassette according to claim 1 or a recombinant plasmid according to claim 10.

12-14. (canceled)

15. A method for the heterologous expression of a gene, characterized in that said method comprises the transfection of a bacterium with an expression cassette according to claim 1.


SEQUENCE LISTING (51 sequences)

SEQ ID NO: 1 - DNA, 29 bp, Bacillus subtilis
ttgtaaagggacaagagctttggtataat

SEQ ID NO: 2 - DNA, 94 bp, Bacillus subtilis
ttgtaaagggacaagagctttggtataatataaaattgtgagtaatagaattattgctccttgcccattatgggccgcttagtccaaaaggagg

SEQ ID NO: 3 - DNA, 113 bp, Bacillus subtilis
gatgcagttgtaaagggacaagagctttggtataatataaaattgtgagtaatagaattattgctccttgcccattatgggccgcttagtccaaaaggaggtgcaaacagatg

SEQ ID NO: 4 - DNA, 20 bp, Bacillus subtilis
  misc_feature (8)..(8): n is a, t, g or c
  misc_feature (13)..(14): n is a, t, g or c
  misc_feature (17)..(18): n is a, t, g or c
  misc_feature (20)..(20): n is a, t, g or c
atttaatntatannatnnan

SEQ ID NO: 5 - DNA, 33 bp, Bacillus subtilis
  misc_feature (6)..(6): n is a, g, t or c
  misc_feature (11)..(13): n is a, g, t or c
  misc_feature (21)..(21): n is a, g, t or c
  misc_feature (26)..(27): n is a, g, t or c
  misc_feature (30)..(31): n is a, g, t or c
  misc_feature (33)..(33): n is a, g, t or c
ttattnatttnnnatttaatntatannatnnan

SEQ ID NO: 6 - DNA, 33 bp, Bacillus subtilis
ttattcatttccgatttaatgtataggatgcag

SEQ ID NO: 7 - DNA, 43 bp, Bacillus subtilis
aaaagtacatatttcttcaaaggaaaaaagcaaaagatgtttt

SEQ ID NO: 8 - DNA, 154 bp, Bacillus subtilis
aaaagtacatatttcttcaaaggaaaaaagcaaaagatgtttttagctgaaggaaaaatgaaaacgaaagataaaaacagaggctgaaagccatttttaagcgtttttcttttcttgttgcatcatttacaatacatacaaccgcaaggagagg

SEQ ID NO: 9 - DNA, 166 bp, Bacillus subtilis
aaaagtacatatttcttcaaaggaaaaaagcaaaagatgtttttagctgaaggaaaaatgaaaacgaaagataaaaacagaggctgaaagccatttttaagcgtttttcttttcttgttgcatcatttacaatacatacaaccgcaaggagaggaggaatcgcatg

SEQ ID NO: 10 - DNA, 10 bp, Bacillus subtilis
ggtataatat

SEQ ID NO: 11 - DNA, 10 bp, Bacillus subtilis
agttgtaaag

SEQ ID NO: 12 - DNA, 13 bp, Bacillus subtilis
  misc_feature (5)..(9): n is a, g, t or c
aaaannnnntttt

SEQ ID NO: 13 - DNA, 13 bp, Bacillus subtilis
  misc_feature (5)..(9): n is a, g, t or c
ataannnnntttt

SEQ ID NO: 14 - DNA, 13 bp, Bacillus subtilis
  misc_feature (5)..(9): n is a, t, g or c
aatannnnntatt

SEQ ID NO: 15 - DNA, 13 bp, Bacillus subtilis
  misc_feature (5)..(9): n is a, g, t or c
aaaannnnntatt

SEQ ID NO: 16 - DNA, 27 bp, Bacillus subtilis
  misc_feature (5)..(9): n is a, g, t or c
  misc_feature (14)..(14): n represents between 10 and 150 nucleotides that can be a, g, t or c
  misc_feature (19)..(23): n is a, g, t or c
aaaannnnnttttnaaaannnnntttt

SEQ ID NO: 17 - DNA, 30 bp, unknown
  forward primer to amplify the ssb promoter region
gcgaagcttccaaacattgacgaagagtct

SEQ ID NO: 18 - DNA, 31 bp, unknown
  Reverse primer to amplify the ssb promoter region
gctggatcctcggttaagcataagaaagacc

SEQ ID NO: 19 - DNA, 315 bp, unknown
  PCR amplification product using Bacillus subtilis as template source and SEQ ID NO 17 and SEQ ID NO18 as primers
gcttccaaacattgacgaagagtctaaaaaagcagttatcgagcgtttcaacaacgttctgacttctaacggtgcggagatcactggaacaaaggattggggtaaacgtcgtcttgcttacgaaatcaacgatttccgcgacggtttctaccaaatcgtaaacgttcaatctgacgctgcggcagttcaagaatttgaccgtctagctaagatcagtgacgatatcattcgccacattgttgttaaagaagaagaataagcaattttgaaatatataatggtaaaaggtggtctttcttatgcttaaccgaggat

SEQ ID NO: 20 - DNA, 28 bp, unknown
  Forward primer to amplify the rpsF promoter region
gcgaagcttgtgactttgagcggggctt

SEQ ID NO: 21 - DNA, 622 bp, unknown
  PCR amplification product using Bacillus subtilis as template source and SEQ ID NO18 and SEQ ID NO20 as primers
gcttgtgactttgagcggggcttcattcgtgctgagacagttgcttatgaggatcttcttgcgggcggcggtatggcaggagctaaagaggcaggaaaagtccgccttgaagggaaagaatatgtggtccaagacggagatgttattcatttccgatttaatgtataggatgcagttgtaaagggacaagagctttggtataatataaaattgtgagtaatagaattattgctccttgcccattatgggccgcttagtccaaaaggaggtgcaaacagatgagaaagtacgaagttatgtacattatccgcccaaacattgacgaagagtctaaaaaagcagttatcgagcgtttcaacaacgttctgacttctaacggtgcggagatcactggaacaaaggattggggtaaacgtcgtcttgcttacgaaatcaacgatttccgcgacggtttctaccaaatcgtaaacgttcaatctgacgctgcggcagttcaagaatttgaccgtctagctaagatcagtgacgatatcattcgccacattgttgttaaagaagaagaataagcaattttgaaatatataatggtaaaaggtggtctttcttatgcttaaccgaggat

SEQ ID NO: 22 - DNA, 30 bp, unknown
  Reverse primer to amplify the rpsF promoter region
gctggatccatcttcgtcaatgtttgggcg

SEQ ID NO: 23 - DNA, 331 bp, unknown
  PCR amplification product using Bacillus subtilis as template source and SEQ ID NO20 and SEQ ID NO22 as primers
gcttgtgactttgagcggggcttcattcgtgctgagacagttgcttatgaggatcttcttgcgggcggcggtatggcaggagctaaagaggcaggaaaagtccgccttgaagggaaagaatatgtggtccaagacggagatgttattcatttccgatttaatgtataggatgcagttgtaaagggacaagagctttggtataatataaaattgtgagtaatagaattattgctccttgcccattatgggccgcttagtccaaaaggaggtgcaaacagatgagaaagtacgaagttatgtacattatccgcccaaacattgacgaagatgg

SEQ ID NO: 24 - DNA, 26 bp, unknown
  Forward primer to amplify the ywpH promoter region
cccaagctttcaagctgtcaatgccg

SEQ ID NO: 25 - DNA, 27 bp, unknown
  Reverse primer to amplify the ywpH promoter region
cgcggatccgattgaacatgcgattcc

SEQ ID NO: 26 - DNA, 300 bp, unknown
  PCR amplification product using Bacillus subtilus as template source and SEQ ID NO24 and SEQ ID NO25 as primers
gctttcaagctgtcaatgccggaaaaaaaattgagctttcagtggtttgcgtgggatggctcttcctatgtgcgcatgaatacgcaaaactggctgacaaagcaaatctttttccgttttttaaaaagtacatatttcttcaaaggaaaaaagcaaaagatgtttttagctgaaggaaaaatgaaaacgaaagataaaaacagaggctgaaagccatttttaagcgtttttcttttcttgttgcatcatttacaatacatacaaccgcaaggagaggaggaatcgcatgttcaatcggat

SEQ ID NO: 27 - DNA, 27 bp, unknown
  amyE-1 Forward primer
gctctagacgaattatatggatgtgac

SEQ ID NO: 28 - DNA, 30 bp, unknown
  amyE-2 Reverse primer
gcctagagctcggatctccttttccgattg

SEQ ID NO: 29 - DNA, 34 bp, unknown
  Kn-1 Forward primer
caatctgcagtcgcgatgagaatagtgaatggac

SEQ ID NO: 30 - DNA, 28 bp, unknown
  Kn-2 Reverse primer
cgggtacctcaaaatggtatgcgttttg

SEQ ID NO: 31 - DNA, 269 bp, unknown
  artificially made spoVG RBS
gaagatctctcgagggtaccttgatacactaatgcttttatatagggaaaaggtggtgaactcatatgaatcgagcttctagagagctcccatggaactatgtgattacgaaaatatatcccttttccaccacttgagtatacttagctcgaagatctctcgagggtaccttgatacacctaatgcttttatatagggaaaaggtggtgcatggaactatgtgattacgaaaatatatcccttttccaccacttgagtatacttagctc

SEQ ID NO: 32 - DNA, 29 bp, unknown
  spoVG1 primer
gaagatctctcgagggtaccttgatacac

SEQ ID NO: 33 - DNA, 30 bp, unknown
  spoVG2 primer
ctatataaaagcattagtgtatcaaggtac

SEQ ID NO: 34 - DNA, 30 bp, unknown
  spoVG3 primer
ctaatgcttttatatagggaaaaggtggtg

SEQ ID NO: 35 - DNA, 30 bp, unknown
  spoVG4 primer
ctcgattcatatgagttcaccaccttttcc

SEQ ID NO: 36 - DNA, 29 bp, unknown
  spoVG5 primer
gactgcagctcgagggtaccttgatacac

SEQ ID NO: 37 - DNA, 41 bp, unknown
  Gntterm-1 Forward primer
gcggatccaggcctaactaattaacctgtattaaaaacacg

SEQ ID NO: 38 - DNA, 33 bp, unknown
  Gntterm-2 Reverse primer
gctctagagttaaccttctgttgtttgggatag

SEQ ID NO: 39 - DNA, 40 bp, unknown
  B-toxoid3 primer
aactgcagagatctcatatgaagaaaaaatttatttcatt

SEQ ID NO: 40 - DNA, 32 bp, unknown
  Btoxoid2 primer
cgcggatccttaaatagctgttactttgtgag

SEQ ID NO: 41 - DNA, 33 bp, unknown
  rpsF-3 primer
ggaattcctgcaggtgactttgagcggggcttc

SEQ ID NO: 42 - DNA, 24 bp, unknown
  rpsF-4 and rpsF-5 primer
cgtactttctcatatgtttgcacc

SEQ ID NO: 43 - DNA, 28 bp, unknown
  amyEback-1 primer
cccaagctttcgacatggatgagcgatg

SEQ ID NO: 44 - DNA, 28 bp, unknown
  amyEback-2 primer
gcagctcgaggctccggcgcaaatgcag

SEQ ID NO: 45 - DNA, 29 bp, unknown
  amyEfront-1 primer
cgcgagctcaacaaaattctccagtcttc

SEQ ID NO: 46 - DNA, 31 bp, unknown
  amyEfront-2 primer
cggtctagaagtttttaatttgtgtgtttcc

SEQ ID NO: 47 - DNA, 23 bp, unknown
  cmR-1 primer
cgggaattctcatgtttgacagc

SEQ ID NO: 48 - DNA, 30 bp, unknown
  cmR-2 primer
cgcgaagcttcccagtagtaggttgaggcc

SEQ ID NO: 49 - DNA, 41 bp, unknown
  Gntterm-3 Forward primer
gcggatcctacgtaaactaattaacctgtattaaaaacacg

SEQ ID NO: 50 - DNA, 315 bp, unknown
  upstream region of the rpsF gene, indicating the location of the -10 and -35 regions and AT-rich stretches further upstream, as well as the RBS and the start codon of the rpsF gene
ctgagtgtgcaggaattattcatagtgactttgagcggggcttcattcgtgctgagacagttgcttatgaggatcttcttgcgggcggcggtatggcaggagctaaagaggcaggaaaagtccgccttgaagggaaagaatatgtggtccaagacggagatgttattcatttccgatttaatgtataggatgcagttgtaaagggacaagagctttggtataatataaaattgtgagtaatagaattattgctccttgcccattatgggccgcttagtccaaaaggaggtgcaaacagatggaagttactgacgt

SEQ ID NO: 51 - DNA, 312 bp, unknown
  upstream region of the ywpH gene
tcaagctgtcaatgccggaaaaaaaattgagctttcagtggtttgcgtgggatggctcttcctatgtgcgcatgaatacgcaaaactggctgacaaagcaaatctttttccgttttttaaaaagtacatatttcttcaaaggaaaaaagcaaaagatgtttttagctgaaggaaaaatgaaaacgaaagataaaaacagaggctgaaagccatttttaagcgtttttcttttcttgttgcatcatttacaatacatacaaccgcaaggagaggaggaatcgcatgttcaatcaggtcatgcttgtcggacgt